(19) United States
(12) Patent Application Publication
(10) Pub. No.: US 2020/0098300 A1
(43) Pub. Date: Mar. 26, 2020

(54) METHODS AND APPARATUS TO SET A BLUE LIGHT CUTOFF TIME OF AN ELECTRONIC DEVICE

(71) Applicant: Intel Corporation, Santa Clara, CA (US)

(72) Inventors: Chetan Verma, Bangalore (IN); Unnikrishnan Pillai, Bangalore (IN)

(21) Appl. No.: 16/484,050

(22) PCT Filed: Apr. 19, 2017
(86) PCT No.: PCT/US17/28312

§ 371 Date: Aug. 6, 2019

(51) Int. Cl. G09G 3/20; G16H 20/30

(52) U.S. Cl. CPC G09G 3/2003; G09G 2380/08; G09G 2320/0666; G16H 20/30

(57) ABSTRACT

Example methods, apparatus, systems, and articles of manufacture to set a blue light cutoff time of an electronic device are disclosed herein. An example electronic device disclosed herein includes a display screen to emit first light in a first mode and second light in a second mode. The first light includes light having a characteristic that negatively affects sleep and the second light has less of the light having the characteristic that negatively affects sleep than the first light. The example electronic device also includes a display color adjuster to set a blue light cutoff time based on sleep data received from a sleep tracking device, and the display is to switch from the first mode to the second mode at the blue light cutoff time.

### FIELD OF THE DISCLOSURE

This disclosure relates generally to electronic devices and, more particularly, to methods and apparatus to set a blue light cutoff time of an electronic device.

### BACKGROUND

People often use their electronic devices such as cellphones, laptops, tablets, etc. at night before going to sleep. For instance, people often lay in bed reading emails, reading news articles, playing games, texting, etc. before going to sleep. The display screens of these devices have a large blue light content. It has been shown that visual exposure to blue light during the night (or before going to sleep) can prolong a person's time-to-fall asleep due to reduction in production of melatonin, which can negatively affect the person's sleep duration and quality of sleep.

### BRIEF DESCRIPTION OF THE DRAWINGS

FIG. 1 illustrates an example system including an example blue light adjuster to set a blue light cutoff time of an example electronic device.

FIG. 2 illustrates an example interface of the example blue light adjuster displayed on the example electronic device of FIG. 1.

FIG. 3 is a flowchart representative of example machine readable instructions that may be executed to implement the example blue light adjuster of FIG. 1.

FIG. 4 is a processor platform that may execute the example instructions of FIG. 3 to implement the example blue light adjuster of FIG. 1.

The figures are not to scale. Wherever possible, the same reference numbers will be used throughout the drawing(s) and accompanying written description to refer to the same or like parts. As used in this patent, stating that any part is in any way positioned on (e.g., positioned on, located on, disposed on, or formed on, etc.) another part, means that the referenced part is either in contact with the other part, or that the referenced part is above the other part with one or more intermediate part(s) located therebetween. Stating that any part is in contact with another part means that there is no intermediate part between the two parts.

### DETAILED DESCRIPTION

Many electronic devices, such as smart phones, laptops, etc. have screens that emit light that is rich in short wavelengths. As such, these devices produce higher concentrations of blue light than natural light to enable a user to see the details on the screen in higher lit areas (e.g., outside in the sun). Blue light is beneficial in that it helps the screen appear crisp and bright. However, studies have shown that blue light affects the level of sleep-inducing hormone melatonin more than any other wave length. In particular, blue light mimics the brightness of the sun, which causes the brain to stop producing melatonin. As a result, blue light can affect the sleep patterns of a person, making it harder to fall and stay asleep. In other words, the blue light may trick a human brain into thinking it is daytime, which interferes with the body's production of melatonin. This lack of melatonin can affect the circadian rhythm of a person, making it harder to fall asleep. Studies have shown that disrupted sleep patterns and/or circadian rhythms can lead to many negative health related issues. For example, poor sleep has been shown to decrease a person's ability to learn and retain new information. Further, poor sleep has been shown to lead to heart disease, obesity, depression and/or other negative health conditions. Thus, good sleep is important as it has many health benefits.

Some electronic device manufacturers and software providers have taken steps to cut off or reduce the blue light portion of the display based on the device's clock setting and geographic location (e.g., based on sunset time). Such steps result in users not being exposed to the blue light during night. In some instances, the electronic device is switched into a different mode (e.g., a sleep mode) that changes the color scheme or tone of the display screen so that less or no blue light is emitted. However, each user may have a different sleep time and pattern. Therefore, applying the same time settings for every individual is a suboptimal solution. Such known solutions do not include a mechanism to provide feedback on sleep quality and, thus, do not adjust the setting in view of user response.

Disclosed herein are example methods, apparatus, systems, and articles of manufacture that set the blue light cutoff schedule of an electronic device based on a user's sleep pattern or behavior. In particular, example methods, apparatus, systems, and articles of manufactured disclosed herein track a user's sleep and set the blue light cutoff time and/or duration on the user's electronic device based on the quality of the user's sleep. Setting or establishing a blue light cutoff time and/or duration may include adjusting the blue light cutoff time and/or duration (e.g., preponing or postponing the time, lengthening or shortening the duration, etc.), not changing the time and/or duration, entering a new time and/or a new duration, converting a prior time and/or a prior duration, overwriting a prior time and/or a prior duration, etc. As used herein, the terms “blue light cutoff time” and “blue light cutoff start time” are used interchangeably and are defined to mean the time at which the blue light content of a display screen is turned off and/or otherwise intentionally reduced (e.g., by switching the electronic device into a different display mode) to reduce the negative effect(s) of the blue light on sleep. For example, the blue light cutoff time may be preponed (moved forward in time) so that the user is exposed to less blue light before the next sleep session, thereby decreasing the negative effects of blue light on sleep as discussed above.

An example blue light adjuster (also referred to as a display color adjuster) disclosed herein analyzes sleep data to determine a quality of sleep of the user and sets (e.g., adjusts) the blue light cutoff time based on the determined quality of sleep. In some examples, the blue light adjuster is implemented as an application or software program executed on the electronic device (e.g., an application on a smart phone). The blue light adjuster receives sleep data from one or more sleep tracking devices, such as a wearable sleep tracker and/or a non-wearable sleep tracker (e.g., a bed-side or in-bed sleep tracker). The sleep tracking device(s) obtain information from the user indicative of sleep, such as movement, heart rate, temperature, etc. The sleep data may include one or more metrics, such as a sleep duration value, a time-to-fall asleep value, a sleep quality value, etc., which may be used to determine whether the user received a good night's sleep. In some examples, the sleep tracking device(s) transmit the sleep data to the blue light adjuster wirelessly after a sleep session (e.g., in the morning).

In some examples, if the blue light adjuster determines the-user did not receive quality sleep, the blue light adjuster may prepone (move to an earlier time) the blue light cutoff time. In other words, the blue light from the electronic device may be negatively affecting the user's sleep. To counteract such a result, the blue light adjuster changes the blue light cutoff time on the electronic device. As a result, before the next sleep session (e.g., before the next night), the blue light content on the screen is reduced or eliminated at an earlier time, which enables the user to fall asleep quicker and enables the user to receive better sleep.

In some examples, the blue light adjuster determines the quality of sleep based on a time-to-fall asleep value, which is the time between when the user intends to go to sleep and the time the user actually falls asleep. The blue light adjuster may compare the time-to-fall asleep value to a time-to-fall asleep threshold. If the time-to-fall asleep value does not satisfy the time-to-fall asleep threshold (e.g., is greater than the time-to-fall asleep threshold), the blue light adjuster changes the blue light cutoff time by preponing the time for the next sleep session such that the user is exposed to less blue light before attempting to go to sleep. Additionally or alternatively, the blue light adjuster may determine the quality of sleep based on other parameters, such as a sleep duration value, which is the total time spent sleeping. The blue light adjuster may compare the sleep duration value to a sleep duration threshold. If the sleep duration value does not satisfy the sleep duration threshold (e.g., is less than the sleep duration threshold), the blue light adjuster may change the blue light cutoff time by preponing the time for the next sleep session. In some examples, the blue light adjuster may determine the quality of sleep based on a sleep quality score, which may be generated by the sleep tracker(s). The blue light adjuster may compare the sleep quality score to a sleep quality threshold. If the sleep quality score does not satisfy the sleep quality threshold (e.g., is less than the sleep quality threshold), the blue light adjuster may change the blue light cutoff time by preponing the time for the next sleep session. Such a reduction in blue light exposure helps reduce interference with melatonin production that would otherwise result from looking at a screen without blue light reduction. Therefore, example methods, apparatus, systems and articles of manufacture disclosed herein help a user achieve better sleep and, thus, may benefit the health of the person.

While examples disclosed herein refer to cutting off or reducing blue light, in some examples, other wavelength light emitted from the electronic device(s) may also be eliminated or reduced. For instance, blue light generally has a wavelength of 440-490 nanometers (nm). In some examples, light having a wavelength less than 500 nm (e.g., including blue light, indigo light, violet light etc.) may also be eliminated or reduced. As such, other wavelength light (besides blue light) that may negatively impact a user's sleep may also be eliminated or reduced.

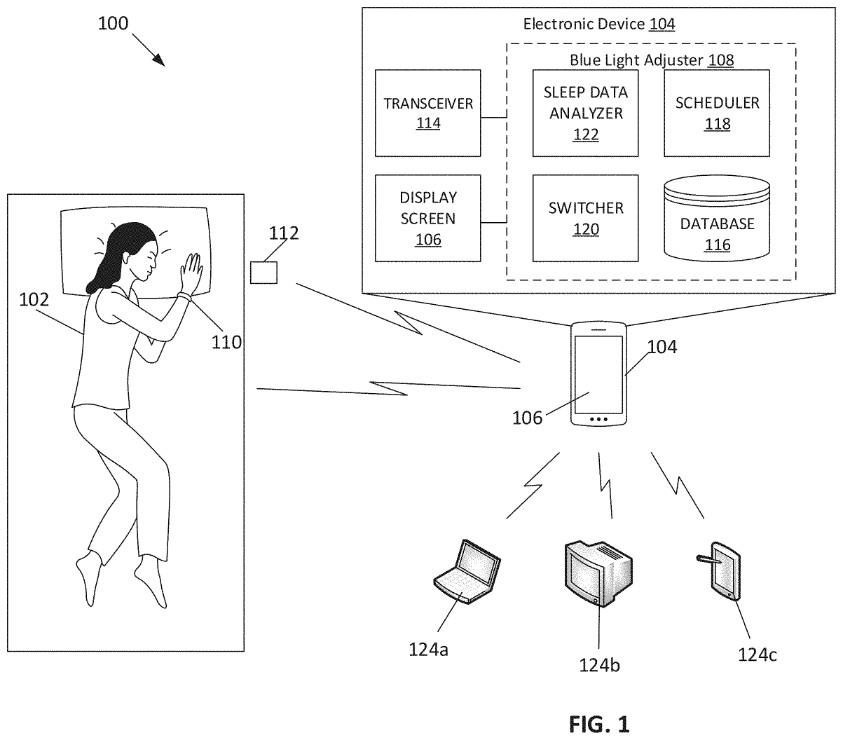

Turning now to the figures, FIG. 1 illustrates an example sleep monitoring system 100. In the illustrated example, a user 102 has an electronic device 104 that he/she may use before going to sleep. In the illustrated example, the electronic device 104 is implemented as a smart phone. However, in other examples, the electronic device 104 may be any other electronic device such as a tablet, a laptop, a personal computer, a watch, a smart television (TV) and/or any other device with a screen that emits blue light. In some examples, the user 102 may have multiple electronic devices. In some examples, the electronic device(s) and/or one or more sleep tracker(s) (disclosed in further detail herein) may be synchronized with a remote server (e.g., one or more devices resident in a “cloud”, such as the iCloud®), over a network such as the internet, which enables all the user device(s) and/or sleep tracker(s) to communicate (directly or indirectly) so that a blue light cutoff schedule can be updated in a synchronized fashion among the electronic device(s). The electronic device 104 is represented twice in FIG. 1, once as a handheld device and once by a block diagram. In the illustrated example, the electronic device 104 includes a display screen 106 that emits light, a portion of which may be blue light. The electronic device 104 is capable of switching display modes and/or otherwise reducing or eliminating the amount of blue light emitted from the display screen 106. For example, the display screen 106 may emit first light in a first mode and second light in a second mode, where the first light has more blue light and the second light has less blue light (e.g., none) than the first light. In some examples, switching from the first mode to the second mode may reduce other light having a characteristic that negatively affects sleep (e.g., shorter wavelength light including blue light) in addition to or as an alternative to the blue light. For example, in the second mode, less light having a wavelength of less than a threshold wavelength may be emitted than in the first mode. For instance, in the second mode, the electronic device 104 may cause the display screen 106 to emit less light having a wavelength less than 500 nanometers (nm) (e.g., blue light, indigo light, violet light, etc.) than in the first mode. In other examples, another threshold wavelength may be used. Thus, any wavelength light (e.g., shorter wavelength light) that may interfere with the user's sleep (e.g., by affecting production of melatonin) is reduced or eliminated. Therefore, wherever blue light is referred to in the specification as being eliminated or reduced, it is understood that other wavelength light that may negatively affect sleep may similarly be eliminated or reduced in addition to or as an alternative to the blue light wavelength. The electronic device 104 may switch back-and-forth between the first and second modes. As mentioned above, it is desirable to have the blue light on when using the electronic device 104 to cause the images on the display screen 106 to appear clearer and crisper. However, the presence of blue light can negatively affect the user's sleep by interfering with the production of melatonin. Therefore, the electronic device 104 is structured to reduce blue light emission at a blue light cutoff time to thereby reduce interference with sleep.

To set (e.g., adjust) the blue light cutoff time of the display screen 106, the example electronic device 104 includes an example blue light adjuster 108 (which may also be referred to as a display color adjuster). The blue light adjuster 108 may be implemented as an application or software program executed by a processor of the electronic device 104. The example blue light adjuster 108 of this example modifies a blue light cutoff time and/or blue light cutoff duration (e.g., a time period beginning with the blue light cutoff time and lasting some duration corresponding to, for instance, an expected sleeping period) based on the sleep pattern/behavior of the user 102. While in the illustrated example the blue light adjuster 108 is implemented in the electronic device 104, in other examples, the blue light adjuster 108 may be implemented on a remote server (e.g., one or more electronic devices located in a “cloud”) that communicates (e.g., over a network such as the internet) with the electronic device 104, one or more other electronic device(s) (e.g., the electronic device(s) 124a, 124b, 124c disclosed in further detail here) and/or one or more sleep tracker(s) (e.g., the sleep tracker(s) 110, 112 disclosed in further detail herein). The remote server may store historical sleep data obtained by the sleep tracker(s). In some examples, the remote server uses one or more data analytics and/or one or more machine learning algorithms to set or establish the blue light cutoff time of a particular user (e.g., via software executed by the remote server).

To determine the sleep pattern/behavior of the user 102, one or more sleep tracking devices are used to obtain sleep data from the user 102 while the user 102 is sleeping. In the illustrated example, the user 102 wears a wearable sleep tracker 110 during sleep. The wearable sleep tracker 110 may be worn around the wrist of the user 102, for example. In other examples, the wearable sleep tracker 110 may be located on other portions of the user's body. In some examples, the wearable sleep tracker 110 is a fitness tracker that also tracks other health related metrics of the user (e.g., calories burned, steps in a day, etc.). In some examples, the fitness tracker provides data on how tired the user 102 is, and the data is used to adjust the blue light cutoff time to enable the user 102 to sleep better. Additionally or alternatively, the sleep monitoring system 100 may include a bedside sleep tracker 112, which is a non-wearable sleep tracking device that may be positioned near the location where the user 102 sleeps (e.g., near a bed). In some examples, the bedside sleep tracker 112 includes a sensor that is to be disposed below a mattress or sleeping surface. In some examples, the sleep tracker is implemented by the electronic device 104 (e.g., by an application (“app”) executing on the device 104).

The sleep tracker(s) 110, 112 include one or more sensors used to gather information indicative of the user's sleep. For example, the sleep tracker(s) 110, 112 may have motion sensors (e.g., an accelerometer), temperature sensors (to detect body temperature), pressure sensors (e.g., to detect the user's pulse), etc. The sleep tracker(s) 110, 112 of this example obtain sleep data from the user while the user is sleeping. In some examples, the sleep data includes a time-to-fall asleep value, which is the length of time between when the user 102 intends to go to sleep and the time the user 102 actually falls asleep. For example, the bedside sleep tracker 112 may determine when the user 102 has laid down to go to sleep using a sensor under the mattress or sleeping surface. Then, based on movement, the bedside sleep tracker 112 can determine when the user 102 actually falls asleep. Likewise, the wearable sleep tracker 110 may be able to determine the time-to-fall asleep value based on movement, pulse, brainwaves, respiration, etc. Additionally or alternatively, the sleep data may include a sleep duration value, which is the total time spent sleeping during the sleep session. The sleep tracker(s) 110, 112 may determine whether the user 102 is sleeping based on movement, body temperature, pulse, and/or other metrics obtained by the sleep tracker(s) 110, 112. For example, when the user 102 hasn't moved for over an hour, the sleep tracker(s) 110, 112 may assume that sleep has begun. When the user 102 wakes up (e.g., in the morning), the movement indicates the user 102 is no longer sleeping. While in the illustrated example the sleep monitoring system 100 includes two sleep trackers 110, 112, in other examples only one of the sleep trackers 110, 112 may be used. In other examples, more than two sleep trackers may be used. Although the example illustrates sleep trackers that actually monitor sleep, in some examples, the sleep tracker(s) 110, 112 lack sensors and, instead, the user 102 (and/or a third party such as a caregiver or a loved one) is to enter the sleep data manually via a user interface. In some examples, the sleep tracker(s) 110, 112 measure melatonin and/or other body parameters, which may be used to set or establish the blue light cutoff time.

To receive the sleep data from the sleep tracker(s) 110, 112, the example electronic device 104 includes an example transceiver 114, which operates as a receiver and a transmitter. The transceiver 114 may be, for example, a Bluetooth® transceiver. In some examples, the sleep tracker(s) 110, 112 are programmed to transmit the sleep data after the user's sleeping session (e.g., in the morning when the user 102 wakes up). For example, the sleep tracker(s) 110, 112 may transmit the sleep data once movement is detected that is indicative of waking up. In other examples, the sleep tracker(s) 110, 112 may constantly (or at a set interval (e.g., every 30 seconds)) transmit the sleep data to the electronic device 104 throughout the sleeping session. The sleep data may be stored in a database 116, for example. In some examples, the database 116 includes data from multiple sleep sessions.

In the illustrated example, the blue light adjuster 108 includes a scheduler 118 that determines and maintains a blue light cutoff schedule for the electronic device 104. The blue light cutoff schedule begins with a blue light cutoff time (e.g., a start time) and extends a duration to an end time. For example, the blue light cutoff schedule may include a blue light cutoff time of 10:00 pm and a blue light cutoff duration of 9 hours (e.g., an end time of 7:00 am). In the illustrated example, the blue light adjuster 108 includes a switcher 120 that interfaces with the display screen 106 to turn the blue light on and/or off (and/or otherwise reduce the amount of blue light). In some examples, the switcher 120 turns on and/or off the blue light and/or otherwise changes the amount of blue light by switching the display screen 106 between the first mode with first light and the second mode with second light. For example, at 10:00 pm, the switcher 120 commands (e.g., via a control signal) the display screen 106 to enter the second mode that emits less (e.g., none) blue light. Then, at 7:00 am, the switcher 120 commands the display screen 106 to switch back to the first mode where the display screen 106 emits more blue light. Additionally or alternatively, the switcher 120 may switch the display screen 106 back to the first mode once the user 102 is awake from sleep. For example, the blue light adjuster 108 may determine the user 102 is awake based on movement detected by the sleep tracker(s) 110, 112 and/or based on user interaction with of the electronic device 104 (e.g., shutting off an alarm, viewing emails, browsing the internet, activating an application (“app”), etc.). Additionally or alternatively, the switcher 120 may alter the amount of blue light (and/or other wavelength light that negatively affects sleep) by modifying the red, green and blue (RGB) values sent to the display screen 106. In some examples, the user 102 may initially enter his/her intended sleep schedule (e.g., 10 pm-7 am) and/or a desired blue light cutoff schedule. In other examples, the blue light adjuster 108 may retrieve historical sleep data and determine the user's sleep schedule automatically. The blue light cutoff time is scheduled some time before the user 102 intends to sleep. Therefore, if the user 102 intends to go to sleep at 11:00 pm, the blue light cutoff time may be set at 10:00 pm.

In the illustrated example of FIG. 1, the example blue light adjuster 108 includes a sleep data analyzer 122 that analyzes the sleep data to determine a quality of sleep for the user 102. Based on the quality of sleep, the scheduler 118 may adjust the blue light cutoff time and/or duration. In some examples, the sleep data analyzer 122 determines the quality of sleep based on a time-to-fall asleep value, which is the length of time between when the user 102 lays down to go to sleep and the time the user 102 actually falls asleep. The user's intent may be inferred by getting into bed or may be explicit based on a user input (e.g., voice recognition of a user saying “I am going to sleep now,” or a selection of a button). For example, the sleep data analyzer 122 may compare the time-to-fall asleep value to a time-to-fall asleep threshold. If the time-to-fall asleep value does not meet the time-to-fall asleep threshold (e.g., is greater than the time-to-fall asleep threshold), the sleep data analyzer 122 determines the user 102 did not receive adequate sleep and the scheduler 118 adjusts the blue light cutoff time. For example, assume the time-to-fall asleep threshold is 30 minutes and the time-to-fall asleep value from the sleep session was 1 hour. In such an instance, the time-to-fall asleep value is greater than the time-to-fall asleep threshold. As such, the scheduler 118 advances (i.e., moves forward in time) the blue light cutoff time. For example, if the original blue light cutoff time (e.g., the first blue light cutoff time) was 10:00 pm, the scheduler 118 may change the blue light cutoff time to 9:30 pm (e.g., the new or second blue light cutoff time). As a result, during the next evening, the blue light content of the display screen 106 is turned off or reduced at the adjusted blue light cutoff time, which is an earlier time than the night before and, thus, reduces the effect of the blue light on the user 102 before going to sleep. Otherwise, if the time-to-fall asleep value meets the time-to-fall asleep threshold (e.g., is less than the time-to-fall asleep threshold), the blue light cutoff time may remain the same (e.g., 10:00 pm) or be postponed (moved later in time), in which case the duration may be shortened. In some examples, in addition to modifying the blue light cutoff time, the scheduler 118 may change (e.g., lengthen) the blue light cutoff duration. For example, if the blue light cutoff time is preponed, the duration may be extended, such that the total blue light cutoff period lasts longer. On the other hand, if the blue light cutoff time is relaxed (moved later in time), the duration may be shortened.

In some examples, in addition to or as an alternative to using the time-to-fall asleep value, the sleep data analyzer 122 may determine the quality of sleep based on a sleep duration value, which is the total time spent sleeping during a sleep session. For example, the sleep data analyzer 122 may compare the sleep duration value to a sleep duration threshold. If the sleep duration value does not meet the sleep duration threshold (e.g., is less than the sleep duration threshold), the sleep data analyzer 122 determines the user 102 did not receive adequate sleep and the scheduler 118 adjusts the blue light cutoff time to reduce blue light earlier in an attempt to increase the sleep duration. For example, assume the sleep duration threshold is 6 hours and the sleep duration value from the previous sleep session was 5.5 hours. The sleep duration value may be low because the user 102 is tossing and turning during sleep, which may be caused by the blue light from the electronic device 104 interfering with the user's quality of sleep. In this example, the sleep duration value is less than the sleep duration threshold. As such, the scheduler 118 prepones the blue light cutoff time. Otherwise, if the sleep duration value is greater than the sleep duration threshold, the blue light cutoff time may remain the same (e.g., 10:00 pm) or be relaxed to a later time.

In some examples, the scheduler 118 only adjusts the blue light cutoff time if both the sleep duration value and the time-to-fall asleep value fail to meet their respective thresholds. In other examples, the scheduler 118 adjusts the blue light cutoff time if at least one of the values fails to meet its respective threshold. While the above examples use data from one night sleep to adjust the blue light cutoff schedule, in some examples, the sleep data analyzer 122 analyzes sleep data from multiple sleep sessions to determine whether to modify the blue light cutoff schedule. For example, the sleep data analyzer 122 may calculate an average of the sleep duration values of the past 5 sleep sessions. If the average is less than the sleep duration threshold, the scheduler 118 may adjust the blue light cutoff time (e.g., prepone the blue light cutoff time 30 minutes). Likewise, multiple nights' sleep data may be used for determining an average time-to-fall asleep value, which may be compared to the time-to-fall asleep threshold and used to adjust the blue light cutoff time. In some examples, using the data from multiple sleep session helps to avoid unnecessarily adjusting the blue light cutoff time due to a single or isolated session of poor sleep.

In some examples, if the time-to-fall asleep value and/or the sleep duration value meet their respective thresholds, the scheduler 118 may postpone the blue light cutoff time (e.g., by 10 minutes). For example, if the sleep data analyzer 122 determines the user 102 is getting a good night sleep, the scheduler 118 may delay the blue light cutoff time to a later time. As such, the blue light adjuster 108 refines the blue light cutoff time to find a time (e.g., an optimal time) that ensures a quality sleep while not affecting the user's ability to use the display screen 106 as desired. Additionally or alternatively, in some examples, if the sleep data analyzer 122 determines that changing the blue light cutoff time is not affecting the sleeping patterns of the user 102, the scheduler 118 may move the blue light cutoff time to a later time. In other words, if the blue light cutoff is not effective for improving sleep, the blue light adjuster 108 avoids unnecessarily adjusting the displayer screen, which may interfere with the sharpness of the display. For example, if the blue light adjuster 108 keeps moving the blue light cutoff time earlier and earlier but the sleep duration value and/or time-to-fall asleep value are not improving, the scheduler 118 may move the blue light cutoff time back to the original blue light cutoff time so as to not unnecessarily interfere with the quality of the display screen 106. This may be referred to as an automatic deactivation of the sleep data analyzer 122.

In some examples, the sleep duration threshold and/or the time-to-fall asleep threshold are stored in the database 116. In some examples, the sleep duration threshold and/or the time-to-fall asleep threshold are based on the user's age and/or medical condition (e.g., if the user 102 has a sleep disorder the threshold may be lower). In some examples, a table of thresholds is stored in the database 116. For example, depending on the age and/or other demographic information about the user 102, the sleep data analyzer 122 may select a particular threshold to use. In other examples, the threshold(s) may be selected by the user 102 (e.g., input into the blue light adjuster 108).

In some examples, whenever the sleep data analyzer 122 determines the quality of sleep is low, the scheduler 118 prepones the blue light cutoff time by a certain increment. For example, if the sleep duration value and/or the time-to-fall asleep value are below their respective thresholds, the scheduler 118 may prepone the blue light cutoff time by 30 minutes. In other examples, the increment may be more (e.g., 1 hour) or less (e.g., 15 minutes). In some examples, the increment may be based on a degree or range of variance from the threshold. For example, if the sleep duration value is between 5 and 6 hours, the scheduler 118 may prepone the blue light cutoff time by 30 minutes, and if the sleep duration value is between 4 and 5 hours, the scheduler 118 may prepone the blue light cutoff time by 1 hour.

In addition to or as an alternative to the sleep duration and time-to-fall asleep values, the sleep data analyzer 122 may determine quality of sleep based on one or more other metrics or body parameters, such as the time spent in rapid eye movement (REM) sleep, temperature of the user 102 (e.g., average temperature throughout the sleep session), heart rate (pulse) of the user 102 (e.g., average heart rate throughout the sleep session), a level of melatonin in the user's body, and/or any other metric indicative of the quality of sleep. The sleep data analyzer 122 may likewise compare the metric(s) to one or more thresholds to determine the quality of sleep and the scheduler 118 may adjust the blue light cutoff time based on the sleep data. In some examples, a sleep quality score or value is generated by one or both of the sleep tracker(s) 110, 112. The sleep data analyzer 122 may compare the sleep quality score to a sleep quality threshold, similar to the time-to-fall asleep value and/or duration values disclosed above, and the scheduler 118 may adjust the blue light cutoff time based on the sleep quality score. The sleep quality score may be based on one or more factors such as, for example, the duration of different sleep stages (e.g., light sleep, deep sleep, REM sleep/dream sleep, etc.), the number of times the user 102 wakes during the sleep session and/or the duration the user 102 is awake, heart rate, breathing rate, skin temperature, snoring level and/or any other metric indicative of the quality of sleep. In some examples, different sleep tracker(s) 110, 112 may calculate the sleep quality score in different manners (e.g., assigning different weights to different factors). In some examples, after every sleep session, the blue light adjuster 108 analyzes the user's pervious sleep data and dynamically modifies the blue light cutoff schedule based on the user's sleep data, thereby refining the blue light cutoff schedule to enable the user to achieve better sleep. In some examples, other data about the user 102 may be used to determine when to adjust the blue light cutoff time. For example, the wearable sleep tracker 110 may be implemented as a fitness tracker that may track how tired the user 102 is throughout the day. This information may be used to adjust the blue light cutoff schedule.

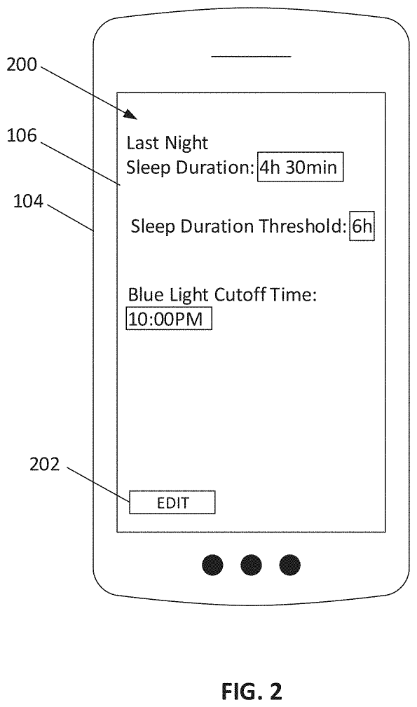

FIG. 2 illustrates an example interface 200 of the example blue light adjuster 108 (FIG. 1) presented on the display screen 106 of the electronic device 104. The interface 200 may display the sleep data (e.g., the sleep duration value, the time-to-fall asleep value, etc.), the results of the analysis, the change in blue light cutoff time, etc. In the illustrated example, the interface 200 shows the user 102 had a sleep duration of 4 hours and 30 minutes, and the sleep duration threshold is set to 6 hours. As such, the scheduler 118 (FIG. 1) advances the blue light cutoff time to an earlier time (e.g., from 10:00 pm to 9:30 pm). In some examples, the user 102 (FIG. 1) may manually enter his/her desired sleep duration threshold into the interface 200. For example, the user 102 may select an edit button 202 that enables the user 102 to modify any of the data in the data fields (e.g., to change the sleep duration threshold, the time-to-fall asleep threshold, the blue light cutoff time, the blue light cutoff duration, etc.). In other examples, the sleep data analyzer 122 (FIG. 1) selects the sleep duration threshold based on the user's weight and/or medical condition.

Referring back to FIG. 1, in some examples, the blue light adjuster 108 communicates with one or more other electronic devices 124a, 124b, 124c to set (e.g., adjust) the blue light schedule on those electronic devices 124, 124b, 124c as well as on the electronic device 104. In other words, the blue light schedule of the electronic device 104 may be synchronized with one or more other electronic devices (e.g., other device(s) associated with the user of the electronic device 104). The other electronic devices 124a, 124b, 124c may include any electronic device such as a smart phone, a tablet, a laptop, a personal computer, a watch, a smart television (TV), etc. The electronic device 104 may transmit, via the transceiver 114, the blue light cutoff schedule (e.g., the blue light cutoff time and/or duration) to the other electronic devices 124a, 124b, 124c either directly or via a remote server. As a result, all of the devices can operate in a manner as to reduce the effects of blue light on the user 102. For example, the electronic device 104 and one or more of the other electronic device(s) 124a, 124b,124c may be synchronized with a remote server (e.g., a virtual machine (VM) in the “cloud”), via a wired or wireless network, such as the internet. In such an example, the electronic device 104 may communicate the blue light schedule to the remote server, which may then communicate the blue light schedule to the other electronic device(s) 124a, 124b, 124c. In some examples, the remote server automatically communicates the blue light schedule to the other electronic device(s) 124a, 124b, 124c when a change is made to the blue light schedule and/or at a scheduled time (e.g., at 6:00 am every day). In other examples, one or all of the other electronic devices 124a, 124b, 124c may include a separate blue light adjuster for the corresponding device. The additional adjuster(s) may operate substantially the same as the blue light adjuster 108.

While in the illustrated example the sleep tracker(s) 110, 112 are depicted as separate from the electronic device 104, in other examples, a sleep tracking device may be integral to the electronic device 104. For example, the electronic device 104 may be a smart watch having a screen (that emits blue light) and which may be worn by the user 102 (e.g., on the wrist of the user 102). The smart watch may include one or more sensors (e.g., a motion sensor, a temperature sensor, etc.) that can be used to obtain sleep data from the user 102 during the sleep session. The blue light adjuster 108 may operate as explained above to adjust the blue light cutoff time and/or duration of the display screen of the watch.

While in the illustrated example the blue light adjuster 108 is implemented in the electronic device 104, in other examples, the blue light adjuster 108 may be implemented on a remote server that communicates (e.g., over a network such as the internet) with the electronic device 104. For example, the electronic device 104 and/or the sleep tracker(s) 110, 112 may transmit the sleep data to the server (or a virtual machine) (e.g., in the “cloud”) that implements the blue light adjuster 108. After the remote blue light adjuster 108 analyzes the sleep data, the blue light adjuster 108 may transmit (e.g., over the internet) the results (e.g., a new blue light cutoff time, no change, etc.) to the electronic device 104. The server may use data analytics and/or a machine learning algorithm to set or establish the blue light cutoff time of a particular user, for example. In other examples, the blue light adjuster 108 may be implemented in one of the sleep trackers 110, 112. After analysis of the sleep data, the sleep tracker(s) 110, 112 may transmit a message to the electronic device 104 to change the blue light cutoff time.

While an example manner of implementing the blue light adjuster 108 is illustrated in FIG. 1, one or more of the elements, processes and/or devices illustrated in FIG. 1 may be combined, divided, re-arranged, omitted, eliminated and/or implemented in any other way. Further, the example database 116, the example scheduler 118, the example switcher 120, the example sleep data analyzer 122 and/or, more generally, the example blue light adjuster 108 of FIG. 1 may be implemented by hardware, software, firmware and/or any combination of hardware, software and/or firmware. Thus, for example, any of the example database 116, the example scheduler 118, the example switcher 120, the example sleep data analyzer 122 and/or, more generally, the example blue light adjuster 108 could be implemented by one or more analog or digital circuit(s), logic circuits, programmable processor(s), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example database 116, the example scheduler 118, the example switcher 120 and/or the example sleep data analyzer 122 is/are hereby expressly defined to include a non-transitory computer readable storage device or storage disk such as a memory, a digital versatile disk (DVD), a compact disk (CD), a Blu-ray disk, etc. storing the software and/or firmware. Further still, the example blue light adjuster 108 of FIG. 1 may include one or more elements, processes and/or devices in addition to, or instead of, those illustrated in FIG. 1, and/or may include more than one of any or all of the illustrated elements, processes and devices.

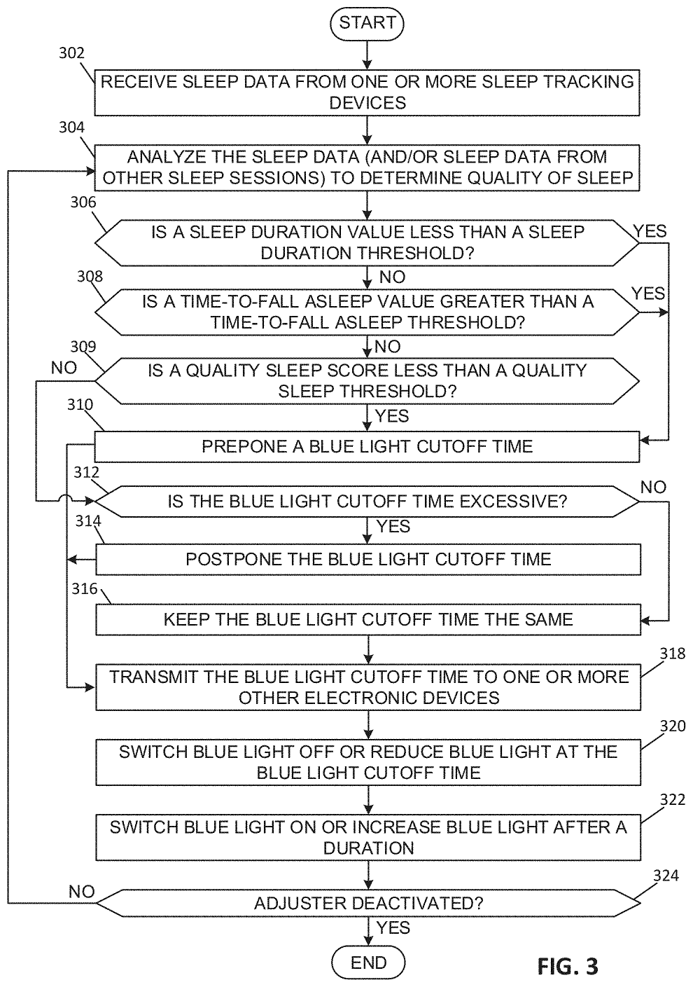

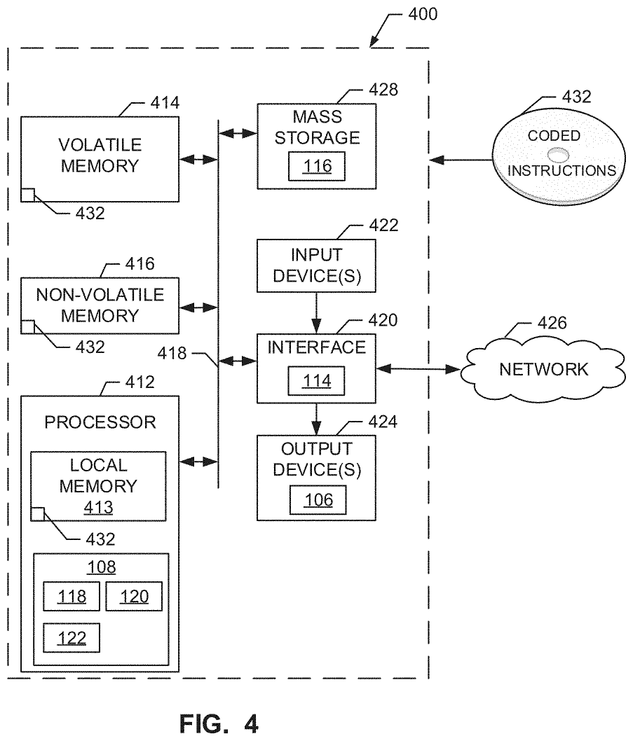

A flowchart representative of example machine readable instructions for implementing the example blue light adjuster 108 of FIG. 1 is shown in FIG. 3. In this example, the machine readable instructions comprise a program for execution by a processor such as the processor 412 shown in the example processor platform 400 discussed below in connection with FIG. 4. The program may be embodied in software stored on a non-transitory computer readable storage medium such as a CD-ROM, a floppy disk, a hard drive, a digital versatile disk (DVD), a Blu-ray disk, or a memory associated with the processor 412, but the entire program and/or parts thereof could alternatively be executed by a device other than the processor 412 and/or embodied in firmware or dedicated hardware. Further, although the example program is described with reference to the flowchart illustrated in FIG. 3, many other methods of implementing the example blue light adjuster 108 may alternatively be used. For example, the order of execution of the blocks may be changed, and/or some of the blocks described may be changed, eliminated, or combined.

As mentioned above, the example process of FIG. 3 may be implemented using coded instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media. As used herein, when the phrase “at least” is used as the transition term in a preamble of a claim, it is open-ended in the same manner as the term “comprising” is open ended. As used herein, “including” and all forms of “including” (e.g., include, includes, etc.) is open ended just as “comprising,” “comprise,” and “comprises” are open ended.

FIG. 3 is a flowchart 300 representative of example machine readable instructions that may be executed by the electronic device 104 to implement the example blue light adjuster 108 of FIG. 1. At block 302, the example blue light adjuster 108 receives (e.g., via the transceiver 114) sleep data from one or more sleep tracking devices, such as the wearable sleep tracker 110 and/or the bedside sleep tracker 112. In some examples, the sleep data is stored in the database 116.

At block 304, the example sleep data analyzer 122 analyzes the sleep data (and/or sleep data from other sleep sessions) to determine a quality of sleep based on the sleep data. Therefore, in the illustrated example of FIG. 1, the sleep data analyzer 122 provides means for analyzing sleep data obtained by a sleep tracking device (e.g., the sleep tracker(s) 110, 112) while a person is sleeping to determine a quality of sleep experienced by the person. In some examples, the sleep data includes a sleep duration value, a time-to-fall asleep value and/or a sleep quality score. In some examples, analyzing the sleep data includes comparing the sleep duration value to a sleep duration threshold, comparing the time-to-fall asleep value to a time-to-fall asleep threshold and/or comparing the sleep quality score to a sleep quality threshold. For example, at block 306, the sleep data analyzer 122 determines if the sleep duration value is less than the sleep duration threshold. At block 308, the sleep data analyzer 122 determines if the time-to-fall asleep value is greater than the time-to-fall asleep threshold. At block 309, the sleep data analyzer 122 determines if the sleep quality score is greater than a sleep quality threshold (assuming the higher the score, the better the quality of sleep). In some examples, only one parameter is analyzed. In other examples, only one of the parameters (and/or additional parameters) are analyzed.

If the sleep duration value is less than the sleep duration threshold, the time-to-fall asleep value is greater than the time-to-fall asleep threshold and/or the sleep quality score is less than the sleep quality threshold, the example scheduler 118 prepones the blue light cutoff time at block 310. In other words, the scheduler 118 changes the blue light cutoff time from a first time (e.g., 10:00 pm) to a second time (e.g., 9:30 pm). Additionally, the blue light cutoff duration or end time may likewise be changed. In some examples, the scheduler prepones the time by a particular time increment (e.g., 30 minutes). In other examples, the scheduler 118 may prepone the blue light cutoff time by different amounts depending on the degree of sleep quality. In some examples, the sleep data (e.g., the time-to-fall asleep value, the sleep duration value, etc.), the threshold(s), and/or the change in blue light cutoff time are displayed on the display screen 106, such as illustrated in FIG. 2.

On the other hand, if the time-to-fall asleep value is at or less than the time-to-fall asleep threshold, the sleep duration value is at or above the sleep duration threshold and the sleep quality score is at or above the sleep quality threshold (e.g., all three conditions are satisfied), the example sleep data analyzer 112 determines if the blue light cutoff time is excessive at block 312. For instance, if the sleep data analyzer 112 108 determines that the user 102 is sleeping in excess of the duration threshold, falling asleep faster (e.g., less than a threshold amount) and/or the sleep quality score is high, the example sleep data analyzer 112 may determine that the blue light cutoff time is an excessive amount of time before the user 102 intends on going to sleep. In some examples, the sleep data analyzer 112 analyzes multiple night's sleep to determine if the user 102 is sleeping longer and/or falling asleep faster. As such, the sleep data analyzer 112 may determine that the blue light cutoff time is unnecessarily early. In such an example, the example scheduler 118 may postpone the blue light cutoff time (e.g., by a particular time increment, such as 30 minutes) to avoid unnecessarily altering the light from the display screen 106, at block 314. Otherwise, if the sleep data analyzer 112 determines that the time between the blue light cutoff time and the time the user 102 intends to go to sleep is not too long, the example scheduler 118 may keep the same blue light cutoff time at block 316. Therefore, in the illustrated example of FIG. 1, the example scheduler 118 provides means for establishing or setting a blue light cutoff time (e.g., preponing the blue light cutoff time, postponing the blue light cutoff time, keeping the blue light cutoff time the same, etc.) of the electronic device 104 based on the quality of sleep.

At block 318, the blue light adjuster 108 transmits (e.g., via the transceiver 114) the blue light cutoff time (and/or duration) to one or more other electronic devices 124a, 124b, 124c. Therefore, in some examples, the example transceiver 114 provides means for transmitting the blue light cutoff time and/or duration to a remote electronic device. At block 320, the example switcher 120 switches the blue light off or reduces the blue light of the display screen 106 (e.g., by sending a control signal to a switch in the electronic device 104) at the blue light cutoff time (e.g., at the preponed, the postponed or the same blue light cutoff time). In some examples, the switcher 120 causes the display screen 106 to enter the second mode, which emits less blue light than in the first mode. Therefore, in the illustrated example of FIG. 1, the switcher 120 provides means for switching the electronic device 104 from the first mode to the second mode and for switching the electronic device 104 from the second mode to the first mode. Additionally or alternatively, the switcher 120 may modify the RGB values transmitted to the display screen 106 to block or reduce the amount of blue light emitted by the display screen 106. At block 322, the switcher 120 switches the blue light back on or increases the blue light (e.g., by switching from the second mode to the first mode, by changing the RGB values transmitted to the display screen 106) after the blue light cutoff duration (e.g., 8 hours). At block 324, the blue light adjuster 108 determines whether the process of FIG. 3 has been deactivated (e.g., via a selection on an interface by the user 102). If the process has not been deactivated, control returns to block 302 and the example process of FIG. 3 repeats again. Otherwise, if the process has been deactivated, the example process of FIG. 3 ends.

FIG. 4 is a block diagram of an example processor platform 400 capable of executing the instructions of FIG. 3 to implement the example blue light adjuster 108 of FIG. 1. The processor platform 400 can be, for example, a server, a personal computer, a mobile device (e.g., a cell phone, a smart phone, a tablet such as an iPad™), a personal digital assistant (PDA), an Internet appliance, a DVD player, a CD player, a digital video recorder, a Blu-ray player, a gaming console, a personal video recorder, a set top box, a smart television (TV) or any other device having some computing capability.

The processor platform 400 of the illustrated example includes a processor 412. The processor 412 of the illustrated example is hardware. For example, the processor 412 can be implemented by one or more integrated circuits, logic circuits, microprocessors or controllers from any desired family or manufacturer. In this example, the processor 412 may implement the example scheduler 118, the example switcher 120, the example sleep data analyzer 122 and/or, more generally, the example blue light adjuster 108.

The processor 412 of the illustrated example includes a local memory 413 (e.g., a cache). The processor 412 of the illustrated example is in communication with a main memory including a volatile memory 414 and a non-volatile memory 416 via a bus 418. The volatile memory 414 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 416 may be implemented by flash memory and/or any other desired type of memory device. Access to the main memory 414, 416 is controlled by a memory controller.

The processor platform 400 of the illustrated example also includes an interface circuit 420. The interface circuit 420 may be implemented by any type of interface standard, such as an Ethernet interface, a universal serial bus (USB), and/or a PCI express interface. In this example, the interface circuit 420 may include the transceiver 114.

In the illustrated example, one or more input devices 422 are connected to the interface circuit 420. The input device(s) 422 permit(s) a user to enter data and commands into the processor 412. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 424 are also connected to the interface circuit 420 of the illustrated example. The output device(s) 424 can be implemented, for example, by display devices (e.g., a light emitting diode (LED), an organic light emitting diode (OLED), a liquid crystal display, and/or a cathode ray tube display (CRT). The interface circuit 420 of the illustrated example, thus, typically includes a graphics driver card, a graphics driver chip or a graphics driver processor. In this example, the output device(s) 424 may include the display screen 106.

The interface circuit 420 of the illustrated example also includes a communication device such as a transmitter, a receiver, a transceiver, a modem and/or network interface card to facilitate exchange of data with external machines (e.g., computing devices of any kind) via a network 426 (e.g., an Ethernet connection, a digital subscriber line (DSL), a telephone line, coaxial cable, a cellular telephone system, etc.).

The processor platform 400 of the illustrated example also includes one or more mass storage devices 428 for storing software and/or data. Examples of such mass storage devices 428 include floppy disk drives, hard drive disks, compact disk drives, Blu-ray disk drives, RAID systems, and digital versatile disk (DVD) drives. In this example, the mass storage devices 428 may include the database 116.

Coded instructions 432 of FIG. 3 may be stored in the mass storage device 428, in the volatile memory 414, in the non-volatile memory 416, and/or on a removable non-transitory computer readable storage medium such as a CD or DVD.

From the foregoing, it will appreciated that methods, apparatus, systems, and articles of manufacture have been disclosed to adjust the blue light cutoff time of an electronic device based on a user's sleep pattern or behavior. Such example methods, apparatus, systems, and articles of manufacture reduce the amount of blue light a user is exposed to before going to sleep, thereby reducing interference of the blue light on the user's level of melatonin and ensuring a better quality sleep. Some example methods, apparatus, systems, and articles of manufacture disclosed herein provide an adaptive feedback system that adjusts the blue light cutoff time without the need of manual input from the user.

Example methods, apparatus/systems and articles of manufacture to adjust a blue light cutoff time of an electronic device are disclosed herein. Further examples and combinations thereof include the following:

Example 1 includes an electronic device including a display to emit first light in a first mode and a second light in a second mode. The first light includes light having a characteristic that negatively affects sleep, and the second light has less of the light having the characteristic that negatively affects sleep than the first light. The example electronic device also includes a display color adjuster to set a blue light cutoff time based on sleep data received from a sleep tracking device, the display to switch from the first mode to the second mode at the blue light cutoff time.

Example 2 includes the electronic device of Example 1, wherein the first light includes light having a wavelength of less than 500 nanometers (nm) and the second light includes less light having a wavelength of less than 500 nm.

Example 3 includes the electronic device of Example 1, wherein the first light includes blue light, and the second light includes less blue light than the first light.

Example 4 includes the electronic device of any of Examples 1-3, wherein the sleep data includes a sleep duration value, and the display color adjuster is to compare the sleep duration value to a sleep duration threshold.

Example 5 includes the electronic device of Example 4, wherein the display color adjuster is to prepone the blue light cutoff time if the sleep duration value is less the sleep duration threshold.

Example 6 includes the electronic device of any of Examples 1-3, wherein the sleep data includes a time-to-fall asleep value, and the display color adjuster is to compare the time-to-fall asleep value to a time-to-fall asleep threshold.

Example 7 includes the electronic device of Example 6, wherein the display color adjuster is to prepone the blue light cutoff time if the time-to-fall asleep value is greater than the time-to-fall asleep threshold.

Example 8 the electronic device of any of Examples 1-3, wherein the sleep data includes a sleep quality score, and the display color adjuster is to compare the sleep quality score to a sleep quality threshold.

Example 9 includes the electronic device of Example 8, wherein the display color adjuster is to prepone the blue light cutoff time if the sleep quality score is less than the sleep quality threshold.

Example 10 includes the electronic device of any of Examples 1-3, wherein the display color adjuster is part of a feedback loop including the sleep tracking device.

Example 11 includes the electronic device of any of Examples 1-3, further including a switcher to cause the display to enter the second mode at the blue light cutoff time.

Example 12 includes the electronic device of any of Examples 1-3, further including a transceiver, the transceiver to receive the sleep data from the sleep tracking device.

Example 13 includes the electronic device of Example 12, wherein the transceiver is to transmit the blue light cutoff time to one or more other electronic devices.

Example 14 includes the electronic device of any of Examples 1-3, wherein the sleep tracking device is integral to the electronic device.

Example 15 includes a method including analyzing, by executing an instruction with at least one processor, sleep data obtained to determine a quality of sleep, the sleep data obtained by a sleep tracking device by monitoring sleep of a user, and setting, by executing an instruction with the at least one processor, a blue light cutoff time of an electronic device based on the quality of sleep.

Example 16 includes the method of Example 15, wherein the sleep data includes a sleep duration value, and the analyzing of the sleep data includes comparing the sleep duration value to a sleep duration threshold.

Example 17 includes the method of Example 16, wherein the setting of the blue light cutoff time includes changing the blue light cutoff time to an earlier time when the sleep duration value is below the sleep duration threshold.

Example 18 includes the method of any of Examples 15-17, wherein the sleep data includes a time-to-fall asleep value, and the analyzing of the sleep data includes comparing the time-to-fall asleep value to a time-to-fall asleep threshold.

Example 19 includes the method of Example 18, wherein the setting of the blue light cutoff time includes changing the blue light cutoff time to an earlier time when the time-to-fall asleep value is greater the time-to-fall asleep threshold.

Example 20 includes the method of Example 15, further including switching, by executing an instruction with the at least one processor, the electronic device from a first mode to a second mode at the blue light cutoff time, wherein in the second mode a display of the electronic device emits less light having a wavelength below 500 nanometers (nm) than in the first mode.

Example 21 includes the method of Example 15, further including transmitting the blue light cutoff time to another electronic device.

Example 22 includes a non-transitory machine readable storage medium including instructions that, when executed, cause at least one machine to at least analyze sleep data obtained by a sleep tracking device while a person is sleeping to determine a quality of sleep experienced by the person and establish a blue light cutoff time of an electronic device based on the quality of sleep.

Example 23 includes the non-transitory machine readable storage medium of Example 22, wherein the sleep data includes a sleep duration value, and the instructions, when executed, cause the at least one machine to analyze the sleep data by comparing the sleep duration value to a sleep duration threshold.

Example 24 includes the non-transitory machine readable storage medium of Example 23, wherein the instructions, when executed, cause the at least one machine to establish the blue light cutoff time by moving the blue light cutoff time.

Example 25 includes the non-transitory machine readable storage medium of any of Examples 22-24, wherein the sleep data includes a time-to-fall asleep value, and the instructions, when executed, cause the at least one machine to analyze the sleep data by comparing the time-to-fall asleep value to a time-to-fall asleep threshold.

Example 26 includes the non-transitory machine readable storage medium of Example 22, wherein the instructions, when executed, cause the at least one machine to adjust the blue light cutoff time by moving the blue light cutoff time to an earlier time.

Example 27 includes the non-transitory machine readable storage medium of Example 22, wherein the instructions, when executed, further cause the at least one machine to switch the electronic device from a first mode to a second mode at the blue light cutoff time, wherein in the second mode a display of the electronic device emits less blue light than in the first mode.

Example 28 includes the non-transitory machine readable storage medium of Example 22, wherein the instructions, when executed, further cause the at least one machine to transmit the blue light cutoff time to a remote electronic device.

Example 29 includes an apparatus including means for analyzing sleep data obtained by a sleep tracking device while a person is sleeping to determine a quality of sleep experienced by the person and means for establishing a blue light cutoff time of an electronic device based on the quality of sleep.

Example 30 includes the apparatus of Example 29, wherein the sleep data includes a sleep duration value, and the means for analyzing is to compare the sleep duration value to a sleep duration threshold.

Example 31 includes the apparatus of Example 30, wherein the means for establishing is to move the blue light cutoff time to an earlier time.

Example 32 includes the apparatus of any of Examples 29-31, wherein the sleep data includes a time-to-fall asleep value, and the means for analyzing is to compare the time-to-fall asleep value to a time-to-fall asleep threshold.

Example 33 includes the apparatus of Example 32, wherein the means for establishing is to move the blue light cutoff time to an earlier time.

Example 34 includes the apparatus of Example 29, further including means for switching the electronic device from a first mode to a second mode at the blue light cutoff time, less light having a wavelength below 500 nanometers (nm) being emitted in the second mode than the first mode.

Example 35 includes the apparatus of Example 29, further including means for transmitting the blue light cutoff time to a remote electronic device.

Although certain example methods, apparatus, systems, and/or articles of manufacture have been disclosed herein, the scope of coverage of this patent is not limited thereto. On the contrary, this patent covers all methods, apparatus and articles of manufacture fairly falling within the scope of the claims of this patent.

1. An electronic device comprising:
a display to emit first light in a first mode and second light in a second mode, the first light including light having a characteristic that negatively affects sleep, the second light having less of the light having the characteristic that negatively affects sleep than the first light; and
a display color adjuster to set a blue light cutoff time based on sleep data received from a sleep tracking device, the display to switch from the first mode to the second mode at the blue light cutoff time.

2. The electronic device of claim 1, wherein the first light includes light having a wavelength of less than 500 nanometers (nm) and the second light includes less light having a wavelength of less than 500 nm.

3. The electronic device of claim 1, wherein the first light includes blue light, and the second light includes less blue light than the first light.

4. The electronic device of claim 1, wherein the sleep data includes a sleep duration value, and the display color adjuster is to compare the sleep duration value to a sleep duration threshold.

5. The electronic device of claim 4, wherein the display color adjuster is to prepone the blue light cutoff time if the sleep duration value is less the sleep duration threshold.

6. The electronic device of claim 1, wherein the sleep data includes a time-to-fall asleep value, and the display color adjuster is to compare the time-to-fall asleep value to a time-to-fall asleep threshold.

7. The electronic device of claim 6, wherein the display color adjuster is to prepone the blue light cutoff time if the time-to-fall asleep value is greater than the time-to-fall asleep threshold.

8-10. (canceled)

11. The electronic device of claim 1, further including a switcher to cause the display to enter the second mode at the blue light cutoff time.

12. The electronic device of claim 1, further including a transceiver, the transceiver to receive the sleep data from the sleep tracking device.

13. The electronic device of claim 12, wherein the transceiver is to transmit the blue light cutoff time to one or more other electronic devices.

14. The electronic device of claim 1, wherein the sleep tracking device is integral to the electronic device.

15. A method including:
analyzing, by executing an instruction with at least one processor, sleep data to determine a quality of sleep, the sleep data obtained by a sleep tracking device by monitoring sleep of a user; and
setting, by executing an instruction with the at least one processor, a blue light cutoff time of an electronic device based on the quality of sleep.

16. The method of claim 15, wherein the sleep data includes a sleep duration value, and the analyzing of the sleep data includes comparing the sleep duration value to a sleep duration threshold.

17. The method of claim 16, wherein the setting of the blue light cutoff time includes changing the blue light cutoff time to an earlier time when the sleep duration value is below the sleep duration threshold.

18. The method of claim 15, wherein the sleep data includes a time-to-fall asleep value, and the analyzing of the sleep data includes comparing the time-to-fall asleep value to a time-to-fall asleep threshold.

19. The method of claim 18, wherein the setting of the blue light cutoff time includes changing the blue light cutoff time to an earlier time when the time-to-fall asleep value is greater the time-to-fall asleep threshold.

20. The method of claim 15, further including switching, by executing an instruction with the at least one processor, the electronic device from a first mode to a second mode at the blue light cutoff time, wherein in the second mode a display of the electronic device emits less light having a wavelength below 500 nanometers (nm) than in the first mode.

21. The method of claim 15, further including transmitting the blue light cutoff time to another electronic device.

22. A non-transitory machine readable storage medium comprising instructions that, when executed, cause at least one machine to at least:
analyze sleep data obtained by a sleep tracking device while a person is sleeping to determine a quality of sleep experienced by the person; and
establish a blue light cutoff time of an electronic device based on the quality of sleep.

23. The non-transitory machine readable storage medium of claim 22, wherein the sleep data includes a time-to-fall asleep value, and the instructions, when executed, cause the at least one machine to analyze the sleep data by comparing the time-to-fall asleep value to a time-to-fall asleep threshold.

24. The non-transitory machine readable storage medium of claim 23, wherein the instructions, when executed, cause the at least one machine to establish the blue light cutoff time by moving the blue light cutoff time to an earlier time.

25. The non-transitory machine readable storage medium of claim 22, wherein the instructions, when executed, further cause the at least one machine to switch the electronic device from a first mode to a second mode at the blue light cutoff time, wherein in the second mode a display of the electronic device emits less blue light than in the first mode.

26. The non-transitory machine readable storage medium of claim 22, wherein the sleep data includes a sleep duration value, and the instructions, when executed, cause the at least one machine to analyze the sleep data by comparing the sleep duration value to a sleep duration threshold.

27. The non-transitory machine readable storage medium of claim 26, wherein the instructions, when executed, cause the at least one machine to establish the blue light cutoff time by moving the blue light cutoff time to an earlier time.

28. The non-transitory machine readable storage medium of claim 22, wherein the instructions, when executed, further cause the at least one machine to transmit the blue light cutoff time to a remote electronic device.
